(19) United States
(12) Patent Application Publication
(10) Pub. No.: US 2010/0112006 A1
(43) Pub. Date: May 6, 2010

(54) Compositions of activated botulinum holotoxin type B (150 kD)

(75) Inventors: Sheryl Ann Garcia, San Jose, CA (US); Bret Marin, Castro Valley, CA (US); Shaji Joseph, Sunnyvale, CA (US)

Correspondence Address: MORGAN, LEWIS & BOCKIUS LLP, 1701 MARKET STREET, PHILADELPHIA, PA, 19103-2921, US

(73) Assignee: Solstice Neurosciences, Inc., Malvern, PA (US)

(21) Appl. No.: 12/590,154

(22) Filed: Nov. 3, 2009

Related U.S. Application Data
(60) Provisional application No. 61/198,106, filed on Nov. 3, 2008.

(51) Int. Cl. A61K 39/08; C12P 21/06

(52) U.S. Cl. 424/239.1; 435/68.1

(57) ABSTRACT

The present invention relates to pharmaceutical compositions of activated botulinum holotoxin type B (150 kD). In particular, the present invention relates to botulinum toxin type B pharmaceutical compositions wherein at least 90% of said botulinum toxin type B is activated (i.e., “nicked”), and wherein at least 99% said nicked botulinum toxin type B is a 150 kD holotoxin (i.e., “stripped”). The invention also relates to a process of activating and stripping botulinum toxin type B wherein at least 90% of said botulinum toxin type B is nicked, and wherein at least 99% of said nicked botulinum toxin type B is stripped. The invention further relates to methods for the treatment of a variety of neuromuscular diseases, pain, inflammatory and cutaneous disorders comprising administering a pharmaceutical composition of activated botulinum holotoxin type B (150 kD) wherein at least 90% of said botulinum toxin type B is nicked, and wherein at least 99% of said nicked botulinum toxin type B is stripped.

### STATEMENT OF RELATED APPLICATIONS

This application claims the benefit of U.S. Patent Application No. 61/198,106 filed on Nov. 3, 2008 which is incorporated herein by reference in its entirety.

### FIELD OF THE INVENTION

The present invention relates to pharmaceutical compositions of activated botulinum holotoxin type B (150 kD). In particular, the present invention relates to botulinum toxin type B pharmaceutical compositions wherein at least 90% of said botulinum toxin type B is activated (i.e., “nicked”), and wherein at least 99% said nicked botulinum toxin type B is a 150 kD holotoxin (i.e., “stripped”). The invention also relates to a process of activating and stripping botulinum toxin type B wherein at least 90% of said botulinum toxin type B is nicked, and wherein at least 99% of said nicked botulinum toxin type B is stripped. The invention further relates to methods for the treatment of a variety of neuromuscular diseases, pain, inflammatory and cutaneous disorders comprising administering a pharmaceutical composition of activated botulinum holotoxin type B (150 kD) wherein at least 90% of said botulinum toxin type B is nicked, and wherein at least 99% of said nicked botulinum toxin type B is stripped.

### BACKGROUND OF THE INVENTION

The anaerobic, gram positive bacterium Clostridium botulinum produces a potent polypeptide neurotoxin, botulinum toxin, which causes a neuroparalytic illness known as botulism in humans and animals by attacking peripheral endings of motor neurons. Botulinum toxin binds with high affinity to acceptor proteins contained in motor neuron terminal endings, is translocated into the cell body and enzymatically cleaves neurotransmitter proteins leading to blockade of the release of the acetylcholine neurotransmitter.

Seven immunologically distinct botulinum neurotoxins have been characterized—these being, respectively, botulinum neurotoxin serotypes A, B, C₁, D, E, F and G—each of which is defined by neutralization with serotype-specific antibodies. Although all the botulinum toxin serotypes apparently inhibit release of the neurotransmitter acetylcholine at the neuromuscular and neuroglandular junction (parasympathetic autonomic nervous tissue interface with target organs), they do so by affecting different neurosecretory proteins and cleaving these proteins at different amino acid residue sites. Consequently, the different serotypes of botulinum toxin vary in their potency, duration of action, and species sensitivity and severity.

Botulinum toxins are the most lethal natural biological toxins known to man and the cause of toxicity in humans known as botulism. The recognition that these toxins could produce muscle paralysis at pharmacologically active does has led to the development of these proteins as a treatment for many human disorders including movement disorders, neuromuscular diseases (e.g., general dystonias, torticollis, hemifacial spasm, bruxism, strabismus, spasticity, cerebral palsy,), as well as sensory disorders (myofascial pain, migraine, tension headaches, neuropathy), autonomic or cutaneous disorders (hyperhydrosis, drooling), and in the treatment of disorders involving inflammation.

Naturally occurring botulinum toxin serotype A is initially synthesized as an inactive single chain proteins which must be cleaved or “nicked” by proteases to become neuroactive, the bacterial strains that make type A possess endogenous proteases. Therefore, the serotype A toxin can be recovered from bacterial cultures in predominantly its active form: approximately 90-95 percent of type A toxin is nicked. In contrast, botulinum toxin serotypes C₁, D and E are synthesized by non-proteolytic strains and are therefore typically unactivated when recovered from culture. Serotypes B and F are produced by both proteolytic and non-proteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. Furthermore, the presence of inactive botulinum toxin molecules in a clinical preparation will contribute to the overall protein load of the preparation, which has been linked to increased antigenicity, without contributing to its clinical efficacy.

The use of a pharmaceutical composition comprising a botulinum toxin type leads to a dose dependent action on nerve terminals that results in irreversible blockade of neurotransmitter release in affect terminal endings of the nerve. The effect is a so-called chemical denervation that results in muscle paralysis when injected into muscles. Recovery from this paralysis occurs by sprouting of immature multiple axon terminals that stabilize the nerve-target organ connection and reverses the denervating effects of the toxin within a period spanning two to six months. Consequently, repeated administration of the neurotoxin is required to maintain a therapeutic effect in a variety of conditions and disorders. However, immunity and resistance to the neurotoxin due to the production of neutralizing antibodies is an important clinical consequence and problem resulting from repeated administrations. For example, the antigenicity of botulinum toxin type A stimulates antibody formation that reduces and most often completely obliterates the therapeutic effectiveness of botulinum toxin type-A-based pharmaceuticals.

The antigenicity of botulinum toxin type A is due in part to its therapeutic administration as part of a botulinum toxin complex. The molecular weight of the botulinum toxin protein molecule, for all seven of the known botulinum toxin serotypes, is about 150 kD. However, the botulinum toxins are released by Clostridial bacterium as complexes comprising the 150 kD botulinum toxin protein molecule—i.e., the “holotoxin”—along with associated non-toxin proteins. The complexes are believed to contain non-toxin hemagglutinin proteins and a non-toxin non-hemagglutinin protein. These non-toxin proteins may act to provide stability against denaturation to the botulinum holotoxin molecule and protection against digestive acids when the toxin is ingested. For example, the toxin complex can be dissociated into toxin protein and hemagglutinin proteins by treating the complex with red blood cells at pH 7.3. The toxin protein has a marked instability upon removal of the hemagglutinin protein. Thus, the botulinum toxin type A complex is naturally produced by Clostridial bacterium as 900 kD, 500 kD and 300 kD complexes. Botulinum toxin types B and C₁ are apparently only produced as 700 kD and 500 kD complexes. Botulinum toxin type D is produced as both 300 kD and 500 kD complexes. Finally, botulinum toxin types E and F are only produced as approximately 300 kD complexes.

Thus, one difficulty with existing pharmaceutical compositions of a botulinum toxin complex is that the presence of the non-toxin proteins contributes to the overall protein load, which has been associated with increased antigenicity, with the potential to diminish clinical efficacy. The size of the complex further limits existing pharmaceutical compositions to be suitable only for intramuscular injection. Additionally, the complex is associated with slower rates of diffusion away from the site of intramuscular injection and thus slower rates of cellular uptake and specific activity. However, the 150 kD holotoxins are unstable and quickly denature when isolated.

### SUMMARY OF THE INVENTION

In some embodiments, a pharmaceutical composition includes botulinum toxin type B and at least one excipient, wherein at least 90% of the botulinum toxin type B is nicked, and wherein at least 99% of said nicked botulinum toxin type B is stripped—i.e., a 150 kD holotoxin.

In some embodiments, a process of activating and stripping botulinum toxin type B includes the stages of: cell growth, activation, purification, and dilution; wherein at least one exogenous protease is administered to a volume of said botulinum toxin type B to increase the level of nicked botulinum toxin type B to at least 90%; and wherein at least one dissociating reagent is administered to a volume of said nicked botulinum toxin type B to increase the level of stripped botulinum toxin type B to at least 99%.

In some embodiments, a method of treating a variety of disorders includes administering to a patient in need thereof, a pharmaceutical composition including activated botulinum holotoxin type B (150 kD) and at least one excipient, wherein at least 90% of said botulinum toxin type B is nicked and wherein at least 99% of said nicked botulinum toxin type B is a 150 kD holotoxin.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 shows an overall manufacturing process flow chart for activated botulinum holotoxin type B (150 kD);

FIG. 2 shows a detailed flow chart for the fermentation stage of the manufacturing process;

FIG. 3 shows a detailed flow chart for the recovery stage of the manufacturing process;

FIG. 4 shows a detailed flow chart for the purification stage of the manufacturing process;

FIG. 5 shows a detailed flow chart for the production and handling of a dilute bulk solution of activated botulinum holotoxin type B (150 kD).

### DETAILED DESCRIPTION OF THE INVENTION

Toxins of the different Clostridium botulinum serotypes are produced in culture as aggregates of neurotoxin and non-toxic proteins non-covalently associated into polypeptide complexes of varying molecular weight. As used herein, “botulinum toxin type B” means an approximately 150 kD protein neurotoxin isolated from the Type B (i.e., Bean strain) of Clostridium botulinum, and associated with non-toxin proteins to form mixtures of its approximately 300-700 kD protein complexes, toxoid, and/or other clostridial proteins, and may refer to either its single-chain or di-chain (“nicked”) neurotoxin form.

As used herein, “botulinum holotoxin type B” means an approximately 150 kD protein neurotoxin isolated from the Type B of Clostridium botulinum, and may refer to either its single-chain or di-chain (“nicked”) neurotoxin form.

As used herein, “activated botulinum holotoxin type B” means a single-chain 150 kD protein type B holotoxin that has undergone limited posttranslational proteolysis (“nicking”) between residues Lys 440 and Ala 441 to form a di-chain protein consisting of an approximately 50 kD light chain linked to an approximately 100 kD heavy chain by a di-sulfide bridge. This nicked form is essential for the neurotoxin's binding to and translocation across epithelial cells at the neuromuscular junction to produce acetylcholine blockage.

According to some embodiments, the present invention describes a pharmaceutical composition of activated botulinum holotoxin type B (150 kD). In some embodiments, the present invention describes a process of activating botulinum toxin type B. In some embodiments, the present invention describes a process of stripping an activated botulinum holotoxin type B (150 kD) from its complex form. And in some embodiments, the present invention describes a method of treating a variety of ophthalmologic disorders, neuromuscular diseases, otorhinolaryngological disorders, urogenital disorders, dermatological disorders, pain disorders, inflammatory disorders, secretory disorders, and cutaneous disorders or cosmetic treatment by administering an effective amount of a pharmaceutical composition of the present invention to a patient in need thereof.

I. Compositions of Activated Botulinum Holotoxin Type B (150 kD)

A. Activated Botulinum Holotoxin Type B (150 kD)

The proteolytic strains that produce the botulinum toxin type B serotype only cleave a portion of the toxin produced: approximately 65% of naturally produced botulinum toxin type B is activated. Further, these proteolytic strains only produce the neurotoxin component (150 kD) of the botulinum toxin type B serotype as part of an approximately 700 kD complex. The present invention discloses a pharmaceutical composition wherein at least 90% of the botulinum toxin type B is activated—i.e., “nicked”—and wherein at least 99% of the nicked botulinum toxin type B is a holotoxin—i.e., the 150 kD neurotoxin component “stripped” from the approximately 700 kD complex.

In some embodiments, the present invention is directed to pharmaceutical compositions of activated botulinum holotoxin type B (150 kD). In some embodiments, at least 90 percent of the botulinum toxin type B in a pharmaceutical composition is nicked. In some embodiments, greater than 90 percent of the botulinum toxin type B in a pharmaceutical composition is nicked. In some embodiments, greater than 90 percent of the botulinum toxin type B in a pharmaceutical composition is nicked. In some embodiments, about 95 percent to about 100 percent of the botulinum toxin type B in a pharmaceutical composition is nicked. In some embodiments, greater than 95 percent of the botulinum toxin type B in a pharmaceutical composition is nicked. In some embodiments, greater than 99 percent of the botulinum toxin type B in a pharmaceutical composition is nicked.

In some embodiments, the activated botulinum toxin type B is stripped of associating proteins and is an activated botulinum holotoxin type B (150 kD). In some embodiments, greater than 99 percent of the botulinum toxin type B in a pharmaceutical composition is stripped. In some embodiments, greater than 99 percent of the botulinum toxin type B in a pharmaceutical composition is stripped.

The increased activation and stripping of botulinum toxin type B in the present invention results in pharmaceutical compositions with comparable efficacy, potency and specific activity to compositions of botulinum type A while limiting the adverse effects of inactive botulinum toxin molecules. Relative to existing pharmaceutical compositions of botulinum toxin type B, the present invention has a decreased overall protein load which results in decreased antigenicity without diminishing clinical efficacy, and may be suitable for transdermal application.

B. Excipients

In some embodiments, the pharmaceutical compositions include activated botulinum holotoxin type B (150 kD), and at least one excipient. As used herein, the term “excipient” means a pharmaceutically acceptable chemical composition, compound, or solvent with which the activated botulinum holotoxin type B (150 kD) may be combined, may stabilize the botulinum toxin and does not alter its physical or therapeutic properties. Excipients suitable for use in the present invention may be selected from the group consisting of, but not limited to: carriers, sequestration agents, surfactants, crystalline agents, buffers, polyaccharides, metals, non-oxidizing amino acid derivatives, sodium chloride, surface active agents, dispersing agents, inert diluents, granulating and disintegrating agents, binding agents, lubricating agents, preservatives, physiologically degradable compositions such as gelatin, aqueous vehicles and solvents, oily vehicles and solvents, suspending agents, dispersing or wetting agents, emulsifying agents, demulcents, salts, thickening agents, fillers, antioxidants, stabilizing agents, and any pharmaceutically acceptable polymeric or hydrophobic materials and other ingredients as known to one of ordinary skill in the art.

1. Sequestration Agents

In some embodiments, a pharmaceutical composition of the present invention includes activated botulinum holotoxin type B (150 kD), and at least one excipient such as a sequestration agent. As used herein, “sequestration agent” means an agent that enhances localization, delivery and/or retention of the botulinum toxin to the site of administration. Examples of proteins, polysaccharides, lipids, polymers, gels and hydrogels that are potentially suitable as sequestration agents are disclosed in U.S. Pat. No. 4,861,627, which is incorporated herein by reference in its entirety. Methods of using and making protein microspheres as sequestration agents, including albumin microspheres, are disclosed in U.S. Pat. Nos. 6,620,617; 6,210,707; 6,100,306; and 5,069,936 which are each incorporated herein by reference in their entirety.

In some embodiments, the sequestration agent is albumin. Human serum albumin may bind with many pharmaceutical agents, including peptides and proteins such as botulinum toxin, which can influence potency, complication rate, clearance, and other pharmacodynamic properties of these agents. Albumin in botulinum toxin pharmaceutical compositions may maintain biologic activity by promoting nerve and other receptor contact and preventing wash out from free neurotoxin release at injection points. Additionally, albumin can non-covalently bind cations that serve as cofactors for enzymatic reactivity of portions of the botulinum toxin polypeptide complex. Specifically, zinc is a cofactor for the endopeptidase activity of the botulinum toxin light chain which enters the target cells after heavy chain binding to the cell surface protein receptors. Higher quantities of zinc bound to albumin enhance endopeptidase activity and thus enhances the denervating effect of botulinum toxin type B. Finally, although other proteins (e.g., gelatin, lactalbumin, lysozyme), lipids and carbohydrates may serve as effective sequestration agents, albumin, including encapsulated albumin and solid microspheres is the preferred protein sequestration agent, in part, because of its low immunogenicity.

2. Buffers

In some embodiments, the excipient is a buffer. In some embodiments, the buffer is succinate. The buffer may be any buffer able to maintain the adequate pH. In some embodiments, the excipient is a buffer to maintain pH from about 5.0 to about 6.0, more preferably from about 5.2 to about 5.8, and most preferably about 5.6.

II. Process of Activating and Stripping Botulinum Toxin Type B

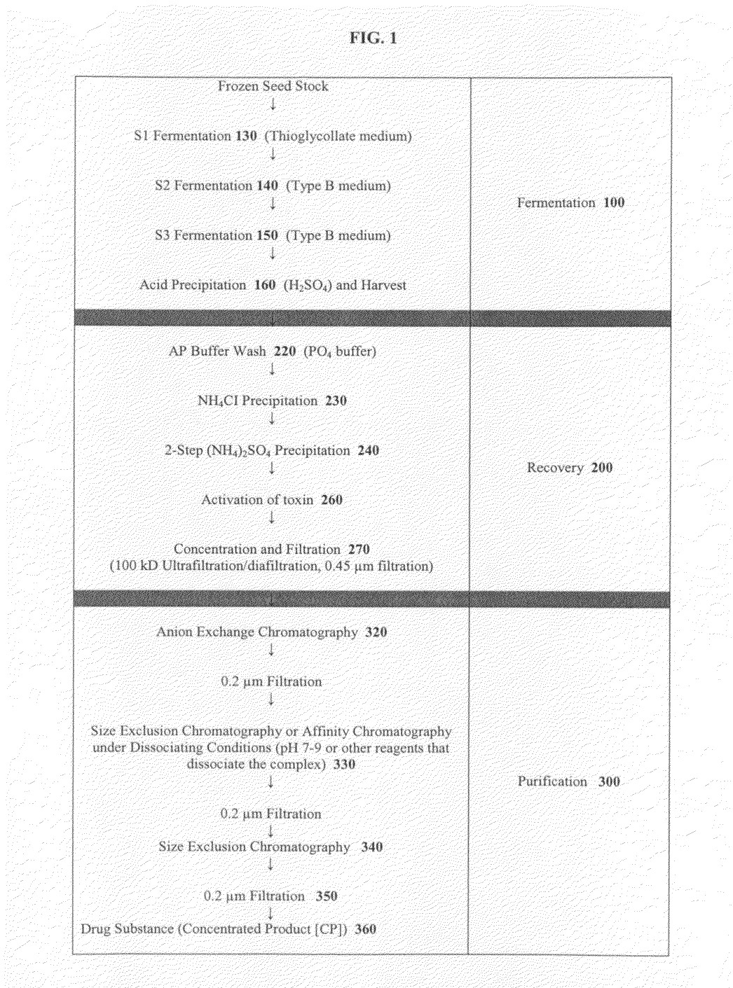

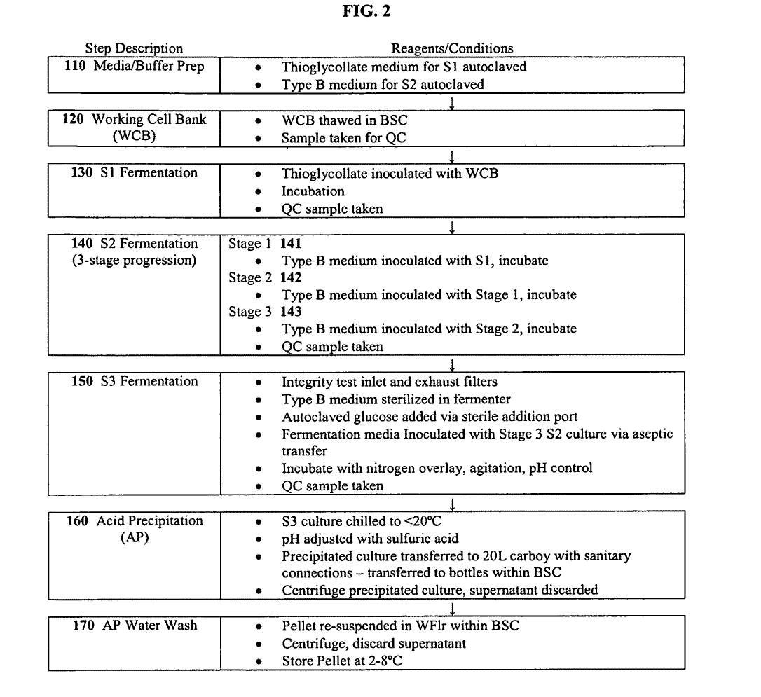

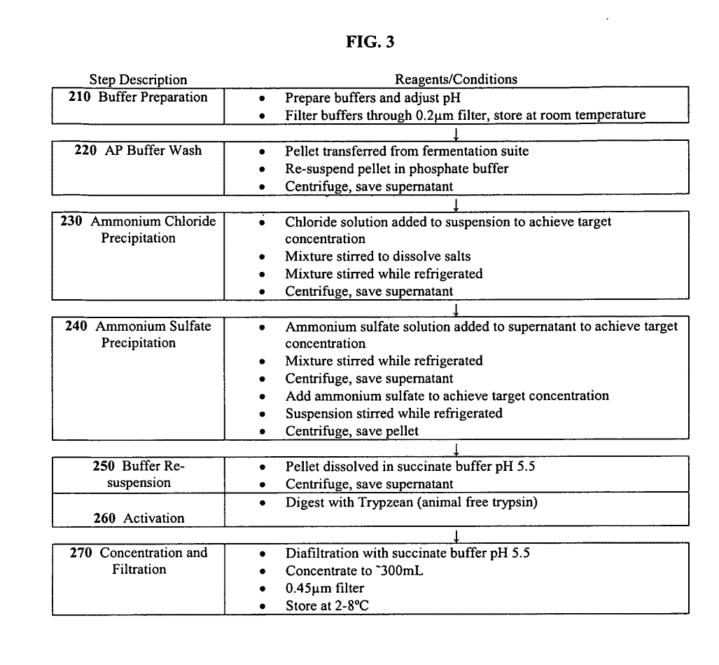

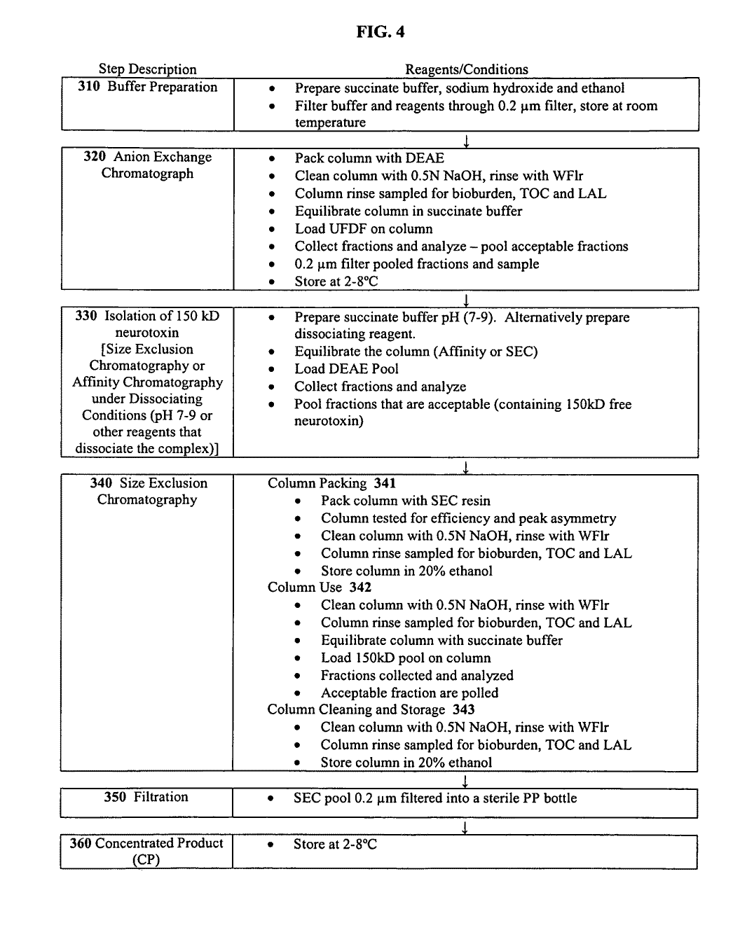

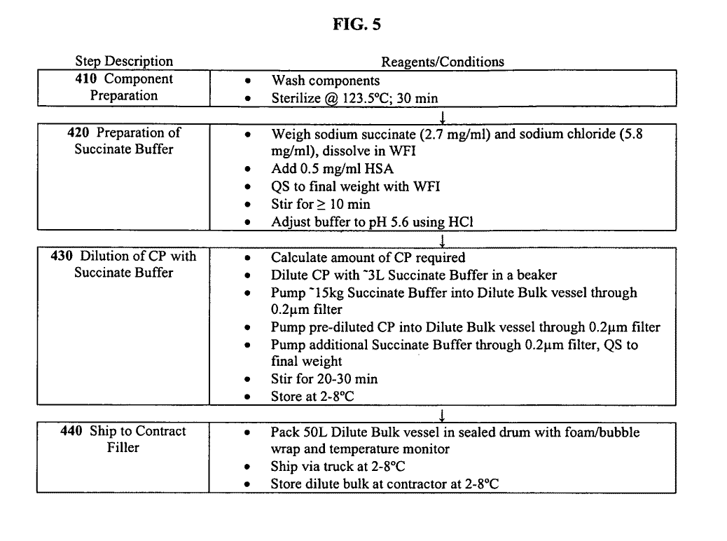

In some embodiments, the present invention describes a process of activating and stripping botulinum toxin type B. Referring to FIG. 1, which shows an overall manufacturing process flow chart for activated botulinum holotoxin type B (150 kD), in some embodiments, a process of activating and stripping botulinum toxin type B according to the present invention may generally be divided into four stages: Fermentation (FIG. 2), Recovery (FIG. 3), Purification (FIG. 4), and Dilute Bulk Solution Preparation (FIG. 5).

A. Fermentation (Cell Growth) Stage

FIG. 2 shows a detailed flow chart for the fermentation or cell growth stage 100 of FIG. 1 of the manufacturing process for activated botulinum holotoxin type B (150 kD). In some embodiments, a process of activating and stripping botulinum toxin type B requires at least one fermentation or cell growth stage 100.

In some embodiments, the fermentation stage 100 includes a media/buffer preparation step 110. In some embodiments, the media buffer preparation step 110 includes autoclaving thioglycollate and Type B mediums for cell growth.

In some embodiments, the fermentation stage 100 includes a working cell bank (WCB) step 120. In some embodiments, the WCB step 120 includes utilizing a frozen culture of Clostridium botulinum, Type B and thawing the frozen culture in a biological safety cabinet (BSC). In some embodiments, the WCB step 120 includes taking a sample of the frozen culture for quality control.

In some embodiments, the fermentation stage 100 includes an S1 fermentation step 130 wherein the autoclaved thioglycollate medium of step 110 is inoculated with the thawed frozen culture of the WCB step 120 and incubated. In some embodiments, the S1 fermentation step 130 includes taking a sample of the resulting S1 cell culture for quality control.

In some embodiments, the fermentation stage 100 includes an S2 fermentation step 140. In some embodiments, the S2 fermentation step 140 includes a three sub-stage progression 141, 142, 143. In some embodiments, the S2 fermentation step 140 includes a first sub-stage 141 wherein the autoclaved Type B medium of step 110 is inoculated with the S1 cell culture of step 130 and incubated. In some embodiments, the S2 fermentation step 140 includes a second sub-stage 142 wherein the autoclaved Type B medium of step 110 is inoculated with the cell culture of the first sub-stage 141 and incubated. In some embodiments, the S2 fermentation step 140 includes a third sub-stage 143 wherein the autoclaved Type B medium of step 110 is inoculated with the cell culture of the second sub-stage 143 and incubated. In some embodiments, the S2 fermentation step 140 includes taking a sample of the resulting cell culture of the third sub-stage 143 for quality control.

In some embodiments, the fermentation stage 100 includes an S3 fermentation step 150. In some embodiments, the S3 fermentation step 150 includes an integrity test and exhaust filters. In some embodiments, the S3 fermentation step 150 includes sterilizing Type B medium in a fermenter. In some embodiments, the S3 fermentation step 150 includes adding autoclaved glucose via a sterile addition port to the sterilized Type B medium. In some embodiments, the S3 fermentation step 150 includes inoculating the sterilized fermentation media with the resulting step 143 cell culture via aseptic transfer. In some embodiments, the S3 fermentation step 150 includes incubating the fermentation medium with a nitrogen overlay, agitation, and pH control of less than pH 6.2. In some embodiments, the S3 fermentation step 150 includes taking a sample of the resulting cell culture for quality control.

In some embodiments, the fermentation stage 100 includes an acid precipitation (AP) step 160. In some embodiments, the AP step 160 includes chilling the S3 cell culture of step 150 to less than 20° C. In some embodiment, the AP step 160 includes adjusting the pH of the step 150 fermentation medium with sulfuric acid. In some embodiments, the AP step 160 includes precipitating the cell culture out of the medium and transferring the cell culture to a 20 L carboy with sanitary connection and subsequent transfer to bottles within BSC. In some embodiments, the AP step 160 includes centrifuging the precipitated cell culture and discarding the supernatant.

In some embodiments, the fermentation stage 100 includes an AP water wash step 170. in some embodiments, the AP water wash step 170 includes re-suspending the centrifuged pellet of step 160 in sterile water for irrigation within the BSC. In some embodiments, the AP water wash step 170 includes centrifuging the re-suspended cell culture and discarding the supernatant. In some embodiments, the AP water wash step 170 includes storing the centrifuged pellet at about 2-8° C.

B. Recovery (Activation) Stage

FIG. 3 shows a detailed flow chart for the recovery or activation stage 200 of FIG. 1 of the manufacturing process for activated botulinum holotoxin type B (150 kD). In some embodiments, a process of activating and stripping botulinum toxin type B requires at least one recovery or activation stage 200. As inactive toxin exhibits the same process chemistry as the activated toxin, an active toxin cannot be seperated from a mixture of active and inactive toxins using simple purification methods. Activation may be performed by the addition of controlled amounts of a proteolytic agent. Activation is controlled by the addition of pre-determined amounts of a proteolytic enzyme and incubating the mixture for a limited time under controlled temperature, pH and mixing.

In some embodiments, the recovery stage 200 includes a buffer preparation step 210. In some embodiments, the buffer preparation step 210 includes preparing and adjusting the pH of phosphate buffers. In some embodiments, the buffer preparation step 210 includes filtering the buffers through a 0.2 μm filter, and storing the filtered buffer at room temperature.

In some embodiments, the recovery stage 200 includes an AP buffer wash step 220. In some embodiments, the AP buffer wash step 220 includes transferring the centrifuged pellet of step 170 from the fermentation suite and re-suspension of the pellet in the phosphate buffer of step 210. In some embodiments, the AP buffer wash step 220 includes centrifugation of the re-suspended pellet and saving the supernatant.

In some embodiments, the recovery stage 200 includes an ammonium chloride precipitation step 230. In some embodiments, the precipitation step 230 includes adding an ammonium chloride solution to the suspension of step 210 to achieve target concentration. In some embodiments, the precipitation step 230 includes stirring the mixture while refrigerated to dissolve salts. In some embodiments, the precipitation step 230 includes centrifuging the mixture and saving the supernatant.

In some embodiments, the recovery stage 200 includes an ammonium sulfate precipitation step 240. In some embodiments, the precipitation step 240 includes adding a solution of ammonium sulfate to the supernatant of step 230 to achieve target concentration. In some embodiments, the precipitation step 240 includes stirring the mixture while refrigerated. In some embodiments, the precipitation step 240 includes centrifuging the mixture and saving the supernatant. In some embodiments, the precipitation step 240 includes adding a second solution of ammonium sulfate to the precipitate to achieve target concentration. In some embodiments, the precipitation step 240 includes stirring the suspension while refrigerated. In some embodiments, the precipitation step 240 includes a second centrifugation and saving the pellet.

In some embodiments, the recovery stage 200 includes a buffer re-suspension step 250. In some embodiments, the re-suspension step 250 includes dissolving the pellet of step 240 in a succinate buffer of pH 5.5. In some embodiments, the re-suspension step 250 includes centrifuging the suspension and saving the supernatant.

In some embodiments, the recovery stage 200 includes an activation step 260. In some embodiments, the activation step 260 includes addition of a protease to the supernatant of step 250. In some embodiments, the protease administered is selected from the group consisting of: trypsin, immobilized TPCK-trypsin, metalloproteases, endogenous proteases, plant derived proteases, bacterial proteases, and gastric proteases. In some embodiments, the protease is an animal free trypsin. In some embodiments, the animal free trypsin used is TrypZean™ (distributed by Sigma-Aldrich®). In some embodiments, the toxin to TrypZean™ ratio is 1:20 to 1:50 (w/w) of toxin.

In some embodiments, the pH range during the activation step 260 is about pH 5 to about pH 6. In some embodiments, the pH level is about 5.6. In some embodiments, the incubation time of the activation step 260 is about 15 minutes to about 24 hours. In some embodiments, the temperature condition of the activation step 260 is about room temperature to about 37° C. In some embodiments, the activation step 260 may be terminated by removing the added protease through diafiltration using suitable filters which can retain the toxin while removing the enzyme. In some embodiments, the activation step 260 may be terminated by adding protease inhibitors to the mixture. In some embodiments, termination of the activation step 260 and the nicking process at various time points yields toxin with varying levels of percentage nicking.

In some embodiments, the recovery stage 200 includes a concentration and filtration step 270. In some embodiments, the concentration and filtration step 270 includes diafiltration of the solution of step 260 with a succinate buffer of pH 5.5 to a concentration of about 300 mL. In some embodiments, the concentration and filtration step 270 includes filtering the buffer through a 0.45 μm filter. In some embodiments, the concentration and filtration step 270 includes storing the filtered buffer at about 2-8° C.

C. Purification Stage

FIG. 4 shows a detailed flow chart for the purification stage 300 of FIG. 1 of the manufacturing process for activated botulinum holotoxin type B (150 kD). In some embodiments, a process of activating and stripping botulinum toxin type B includes a purification stage 300.

In some embodiments, the purification stage 300 includes a buffer preparation step 310. In some embodiments, the buffer preparation step 310 includes preparing a succinate buffer, sodium hydroxide, and ethanol. In some embodiments, the buffer preparation step 310 includes filtering the succinate buffer and reagents through a 0.2 μm filter. In some embodiments, the filtered buffer and reagents is stored at room temperature.

In some embodiments, the purification stage 300 includes an anion exchange chromatograph step 320. In some embodiments, the chromatograph step 320 includes packing a chromatograph column with DEAE. In some embodiments, the chromatograph step 320 includes cleaning the column with 0.5 N NaOH and rinsing with filtered water. In some embodiments, the chromatograph step 320 includes sampling the column rinse for bioburden, total organic carbon (TOC) and limulus amebocyte lysate (LAL) for endotoxin testing. In some embodiments, the chromatograph step 320 includes equilibrating the chromatograph column with the succinate buffer of step 310. In some embodiments, the chromatograph step 320 includes loading an ultra-filtration diafiltration (UFDF) on the column. In some embodiments, the chromatograph step 320 includes collecting and analyzing fractions via SDS-PAGE gels. In some embodiments, the chromatograph step 320 includes pooling acceptable fractions. In some embodiments, the chromatograph step 320 includes filtering the pooled fractions through a 0.2 μm filter and sampling the filtered pooled fractions. In some embodiments, the chromatograph step 320 includes storing the filtered pooled fractions at about 2-8° C.

In some embodiments, the purification stage 300 includes an isolation of 150 kD neurotoxin step 330 wherein the holotoxin is “stripped” from the toxin complex. In some embodiments, the isolation step 330 includes preparing a succinate buffer. In some embodiments, the succinate buffer has a pH level of about 7 to about 9. In some embodiments, the isolation step 330 includes alternatively preparing a dissociating reagent. In some embodiments, the isolation step 330 includes equilibrating a column. In some embodiments, the column is a size exclusion chromatography column. In some embodiments, the column is an affinity chromatography column. In some embodiments, the isolation step 330 includes loading the column with the filtered pooled fractions of step 320. In some embodiments, the isolation step 330 includes collecting and analyzing fractions via SDS-PAGE gels. In some embodiments, the isolation step 330 includes pooling fractions that are acceptable—i.e., that contain the 150 kD free holotoxin.

In some embodiments, the purification stage 300 includes a size exclusion chromatography step 340. In some embodiments, the size exclusion chromatography step 340 includes a column packing sub-step 341, a column use sub-step 342, and a column cleaning and storage sub-step 343. In some embodiments, the column packing sub-step 341 includes packing the column with size exclusion chromatography (SEC) resin. In some embodiments, sub-step 341 includes testing the column for efficiency and peak asymmetry. In some embodiments, sub-step 341 includes cleaning the column with 0.5 NaOH and rinsing with filtered water. In some embodiments, sub-step 341 includes sampling the column rinse for bioburden, TOC, and LAL. In some embodiments, sub-step 341 includes storing the column in 20% ethanol.

In some embodiments, the size exclusion chromatography step 330 includes a column use sub-step 342. In some embodiments, sub-step 342 includes cleaning the column with 0.5 NaOH and rinsing with sterile water for irrigation. In some embodiments, sub-step 342 includes sampling the column rinse for bioburden, TOC, and LAL. In some embodiments, sub-step 342 includes equilibrating the column with the succinate buffer of step 310. In some embodiments, sub-step 342 includes loading the filtered pooled fractions of step 330 on the column. In some embodiments, sub-step 342 includes collection and analyzing fractions via SDS-PAGE gels. In some embodiments, sub-step 342 includes pooling acceptable fractions.

In some embodiments, the size exclusion chromatography step 330 includes a column cleaning and storage sub-step 343. In some embodiments, sub-step 343 includes cleaning the column with 0.5 NaOH and rinsing with filtered water. In some embodiments, sub-step 343 includes sampling the column rinse for bioburden, TOC, and LAL. In some embodiments, sub-step 343 includes storing the column in 20% ethanol.

In some embodiments, the purification process 300 includes a filtration step 350. In some embodiments, the filtration step 350 includes filtering the pooled fractions of step 342 through a 0.2 μm filter into a sterile bottle.

In some embodiments, the purification process 300 includes a concentrated product (CP) step 360. In some embodiments, the filtered concentrated product of step 350 is stored at about 2-8° C.

D. Dilute Bulk Solution Preparation Stage

FIG. 5 shows a detailed flow chart for the production and handling of a dilute bulk solution of activated botulinum holotoxin type B (150 kD). In some embodiments, a process for activating botulinum toxin type B includes a dilute bulk solution preparation stage 400.

In some embodiments, the dilute bulk solution preparation stage 400 includes a component preparation step 410. In some embodiments, the component step 410 includes washing and sterilizing the components at 123.5° C. for 30 minutes.

In some embodiments, the dilute bulk solution preparation stage 400 includes a succinate buffer preparation step 420. In some embodiments, the buffer preparation step 420 includes weighing sodium succinate and sodium chloride and dissolving them in filtered water. In some embodiments, the sodium succinate weighed is 2.7 mg/mL. In some embodiments, the sodium chloride weighed is 5.8 mg/mL. In some embodiments, the buffer preparation step 420 includes adding human serum albumin (HSA). In some embodiments, the HSA is 0.5 mg/mL. In some embodiments, the buffer preparation step 420 includes addition of sterile water for injection, stirring, and adjustment of the buffer to a pH of 5.6 using hydrogen chloride.

In some embodiments, the dilute bulk solution preparation stage 400 includes a dilution step 430 of the concentrated product with succinate buffer. In some embodiments, the dilution step 430 includes calculating the amount of the concentrated product (CP) of step 350 required and diluting the CP with the prepared succinate buffer of step 420. In some embodiments, the CP is diluted with about 3 L of succinate buffer. In some embodiments, the dilution step 430 includes pumping about the succinate buffer of step 420 into a dilute bulk vessel through a 0.2 μm filter. In some embodiments, the dilution step 430 includes pumping the pre-diluted CP into a dilute bulk vessel through a 0.2 μm filter. In some embodiments, the dilution step 430 includes pumping additional succinate buffer through the 0.2 μm filter, stirring for 20-30 minutes, and storing the diluted bulk solution at about 2-8° C.

III. Method of Treatment Using Activated Botulinum Holotoxin Type B (150 kD)

The increased percentage of activated botulinum holotoxin type B (150 kD) molecules in a pharmaceutical composition of the present invention enhances the clinical effectiveness of the botulinum toxin, allows for the decreased protein load of a preparation, and results in decreased antigenicity.

The pharmaceutical compositions of the present invention may be administered by any means known in the art to deliver the activated botulinum holotoxin type B (150 kD) to the desired therapeutic target. In some embodiments, the pharmaceutical compositions are delivered by transmucosal administration. In some embodiments, the pharmaceutical compositions are delivered by transcutaneous administration. In some embodiments, the pharmaceutical compositions are delivered by intramuscular administrations. In some embodiments, the pharmaceutical compositions are delivered by transdermal administration. In some embodiments, the pharmaceutical compositions are injection. In some embodiments, the pharmaceutical compositions are delivered topically.

The pharmaceutical compositions of the present invention may be used in any of the methods of treatment disclosed herein. According to the methods disclosed herein, the pharmaceutical compositions of the present invention may be administered as a single treatment or repeated periodically to provide multiple treatments.

In some embodiments, the present invention describes a method of treating a variety of opthalmologic disorders, neuromuscular diseases, otorhinolaryngological disorders, urogenital disorders, dermatological disorders, pain disorders, inflammatory disorders, secretory disorders, and cutaneous disorders or cosmetic treatment by administering an effective amount of a pharmaceutical composition of the present invention to a patient in need thereof. As used herein, an “effective amount” is an amount sufficient to produce a therapeutic response. An effective amount may be determined with dose escalation studies in open-labeled clinical trials or bin studies with blinded trials.

Pharmaceutical compositions according to the invention may be used for preparing medicaments intended to treat a disease, condition, or syndrome may be chosen from, but not limited to, the following:

A. Opthalmologic Disorders

In some embodiments, a method of treating opthalmologic disorders includes administering an effective amount of a pharmaceutical composition of the present invention to a patient in need thereof. In some embodiments, the opthalmologic disorder is selected from the group consisting of, but not limited to: blepharospasm, strabismus (including restrictive or myogenic strabismus), amblyopia, oscillopsia, protective ptosis, theraputic ptosis for corneal protection, nystagmus, estropia, diplopia, entropion, eyelid retraction, orbital myopathy, heterophoria, concomitant misalignment, nonconcomitant misalignment, primary or secondary esotropia or exotropia, internuclear opthalmophegia, skew deviation, Duane's syndrome and upper eyelid retraction

B. Overactive Muscles or Neuromuscular Diseases

As used herein, “overactive muscles or neuromuscular diseases” refer to any disease adversely affecting both nervous elements (brain, spinal cord, peripheral nerve) or muscle (striated or smooth muscle), including but not limited to: involuntary movement disorders, dystonias, spinal cord injury or disease, multiple sclerosis, and spasticity from cerebral palsy, stroke, or other cause.

In some embodiments, a method of treating neuromuscular diseases includes administering an effective amount of a pharmaceutical composition of the present invention to a patient in need thereof. In some embodiments, the neuromuscular disease is an involuntary movement disorder selected from the group consisting of, but not limited to: hemifacial spasm, torticollis, spasticity of the child or of the adult (e.g., in cerebral palsy, post-stroke, multiple sclerosis, traumatic brain injury or spinal cord injury patients), idiopathic focal dystonias, muscle stiffness, writer's cramp, hand dystonia, CN VI nerve palsy, oromandibular dystonia, head tremor, tardive dyskinesia, occupational cramps (including musicians' cramp), facial nerve palsy, jaw closing spasm, facial spasm, synkinesia, tremor, primary writing tremor, myoclonus, stiff-person-syndrome, foot dystonia, facial paralysis, painful-arm-and-moving-fingers-syndrome, tic disorders, dystonic tics, Tourette's syndrome, neuromyotonia, trembling chin, lateral rectus palsy, dystonic foot inversion, jaw dystonia, Rabbit syndrome, cerebellar tremor, III nerve palsy, palatal myoclonus, akasthesia, muscle cramps, IV nerve palsy, freezing-of-gait, extensor truncal dystonia, post-facial nerve palsy synkinesis, secondary dystonia, off period dystonia, cephalic tetanus, myokymia and benign cramp-fasciculation syndrome.

C. Otorhinolaryngological or Gastrointestinal Disorders

In some embodiments, a method of treating otorhinolaryngological or gastrointestinal disorders includes administering an effective amount of a pharmaceutical composition of the present invention to a patient in need thereof. In some embodiments, the otorhinolaryngological disorder is selected from the group consisting of, but not limited to: spasmodic dysphonia, hypersalivation, sialorrhoea, ear click, tinnitus, vertigo, Meniere's disease, cochlear nerve dysfunction, stuttering, cricopharyngeal dysphagia, bruxism, closure of larynx in chronic aspiration, vocal fold granuloma, ventricular dystonia, ventricular dysphonia, mutational dysphonia, trismus, snoring, voice tremor, aspiration, tongue protrusion dystonia, palatal tremor and laryngeal dystonia; gastrointestinal disorders selected from the group consisting of achalasia, anal fissure, constipation, temperomandibular joint dysfunction, sphincter of Oddi dysfunction, sustained sphincter of Oddi hypertension, intestinal muscle disorders, puborectalis syndrome, anismus, pyloric spasm, gall bladder dysfunction, gastrointestinal or oesophageal motility dysfunction, diffuse oesophageal spasm, oesophageal diverticulosis and gastroparesis.

D. Urogenial Disorders

In some embodiments, a method of treating urogenital disorders includes administering an effective amount of a pharmaceutical composition of the present invention to a patient in need thereof. In some embodiments, the urogenital disorder is selected from the group consisting of, but not limited to: detrusor sphincter dyssynergia, detrusor hyperreflexia, neurogenic bladder dysfunction in Parkinson's disease, spinal cord injury, stroke or multiple sclerosis patients, bladder spasms, urinary incontinence, urinary retention, hypertrophied bladder neck, voiding dysfunction, interstitial cystitis, vaginismus, endometriosis, pelvic pain, prostate gland enlargement (Benign Prostatic Hyperplasia), prostatodynia, prostate cancer and priapism.

E. Dermatological Disorders

In some embodiments, a method of treating dermatological disorders includes administering an effective amount of a pharmaceutical composition of the present invention to a patient in need thereof. In some embodiments, the dermatological disorder is selected from the group consisting of, but not limited to: axillary hyperhidrosis, palmar hyperhidrosis, Frey's syndrome, bromhidrosis, psoriasis, skin wounds and acne.

F. Pain Disorders

In some embodiments, a method of treating pain disorders includes administering an effective amount of a pharmaceutical composition of the present invention to a patient in need thereof. In some embodiments, the pain disorder is selected from the group consisting of, but not limited to: joint pain, upper back pain, lower back pain, myofascial pain, tension headache, fibromyalgia, myalgia, migraine, whiplash, joint pain, post-operative pain and pain associated with smooth muscle disorders.

G. Inflammatory Disorders

In some embodiments, a method of treating inflammatory disorders includes administering an effective amount of a pharmaceutical composition of the present invention to a patient in need thereof. In some embodiments, the inflammatory disorder is selected from the group consisting of, but not limited to: pancreatitis, gout, tendonitis, bursitis, dermatomyositis and ankylosing spondylitis.

H. Secretory Disorders

In some embodiments, a method of treating secretory disorders includes administering an effective amount of a pharmaceutical composition of the present invention to a patient in need thereof. In some embodiments, the secretory disorder is selected from the group consisting of, but not limited to: excessive gland secretions, mucus hypersecretion and hyperlacrimation and holocrine gland dysfunction.

I. Cutaneous Disorders or Cosmetic Treatment

In some embodiments, a method of treating cutaneous disorders or cosmetic treatment includes administering an effective amount of a pharmaceutical composition of the present invention to a patient in need thereof. In some embodiments, the cutaneous disorder or cosmetic treatment is selected from the group consisting of, but not limited to: skin defects; facial asymmetry; wrinkles selected from glabellar frown lines and facial wrinkles; downturned mouth; and hair loss.

### EXAMPLES

The following Examples serve to further illustrate the present invention and are not to be construed as limiting its scope in any way.

### Example 1

### Preparation of an Activated Botulinum Holotoxin Type B (150 kD) Composition

### Fermentation (Cell Growth) Stage

The drug substance manufacturing process, which utilizes a frozen culture of C. botulinum, Type B Bean strain (working cell bank), proceeds through two successive seed cultures (S1 and S2). The S2 seed culture is used as the inoculum for the production culture (S3). In S3, a fermentor containing liquid medium of casein hydrolysate (trypticase peptone), yeast extract, cysteine hydrochloride, and glucose is inoculated with an S2 culture. After fermentation, the crude toxin complex is precipitated by acidifying the culture.

### Example 2

### Preparation of an Activated Botulinum Holotoxin Type B (150 kD) Composition

### Recovery (Activation) Stage

The precipitated toxin is re-suspended in phosphate buffer and purified by a series of salt precipitations including 2 M ammonium chloride/0.7 mM magnesium chloride precipitation step, a 15% ammonium sulfate precipitation step and 30% ammonium sulfate precipitation step. The pellet is re-suspended in succinate buffer. The dissolved toxin is digested with TrypZean™ (animal free proteolytic enzyme) to nick and activate the toxin at temperature range of 20° C.-40° C. and pH of 5-6, for a period of 30 min to 120 minute. Upon completion of incubation, the toxin solution is diafiltered to remove solutes and the added proteolytic enzyme, and then filtered (0.45 μm). The activation yields toxin with percentage nicking of >90%.

### Example 3

### Preparation of an Activated Botulinum Holotoxin type B (150 kD) Composition

### Purification Stage

Purification is accomplished using anion exchange, affinity or size exclusion under dissociated conditions (pH 7-9 or other dissociating agents) and size exclusion column (SEC) chromatography as a polishing step at pH 5.5, each followed by 0.2 μm filtration. The concentrated product is produced at the completion of the filtering step from the final SEC column.

### Example 4

### Preparation of an Activated Botulinum Holotoxin Type B (150 kD) Composition

### Dilute Bulk Solution Stage

The concentrated product (CP) is diluted to 5000 U/mL with 10 mM succinate buffer (pH 5.6) containing 100 mM sodium chloride and 0.5 mg Human Serum Albumin (HSA) per mL to prepare the bulk drug product, also named dilute bulk solution. The dilute bulk is 0.2 μm filtered to reduce bioburden and prepared in a 45 L batch size.

### Example 5

### Preparation of an Activated Botulinum Holotoxin type B (150 kD) Composition

### Final Container Preparation

The dilute bulk solution is sterile filtered through two 0.22 μm filters in series prior to filling. Three final product presentations 0.5 mL, 1.0 mL, and 2.0 mL are filled into USP Type I glass vials (3.5 mL). The vials are closed with siliconized butyl rubber stoppers and sealed with aluminum seals. The final product is stored refrigerated at 5±3° C.

The present application incorporates U.S. patent application Ser. No. 12/462,560 herein by reference in its entirety.

What is claimed is:

1. A pharmaceutical composition comprising:
(a) activated botulinum toxin type B; and
(b) at least one excipient;
wherein at least 90% of said botulinum toxin type B is nicked; and
wherein at least 99% of said nicked botulinum toxin type B is an approximately 150 kD holotoxin.

2. The pharmaceutical composition of claim 1, wherein greater than 90 percent of said botulinum toxin B is nicked.

3. The pharmaceutical composition of claim 1, wherein approximately about 95 percent to about 100 percent of said botulinum toxin type B is nicked.

4. The pharmaceutical composition of claim 1, wherein greater than 95 percent of said botulinum toxin B is nicked.

5. The pharmaceutical composition of claim 1, wherein greater than 99 percent of said botulinum toxin B is nicked.

6. The pharmaceutical composition of claim 1, wherein greater than 99 percent of said botulinum toxin B is stripped.

7. The pharmaceutical composition of claim 1, wherein said at least one excipient is selected from the group consisting of: buffers, carriers, stabilizers, preservatives, diluents, vehicles, bulking agents, albumins, gelatins, collagens, proteins, polysaccharides, metals, non-oxidizing amino acid derivatives, and sodium chloride.

8. The pharmaceutical composition of claim 7, wherein said at least one excipient is albumin.

9. The pharmaceutical composition of claim 7, wherein said at least one excipient is a buffer.

10. The pharmaceutical composition of claim 9, wherein said buffer is a succinate buffer.

11. A process of activating and stripping botulinum toxin type B, comprising the stages of:
cell growth, activation, purification, and dilution; wherein at least one protease is administered to a volume of said botulinum toxin type B; wherein said protease administered increases the levels of nicked botulinum toxin type B to at least 90%;
wherein at least one dissociating reagent is administered to a volume of said nicked botulinum toxin type B; and wherein said dissociating reagent administered increases the levels of stripped botulinum toxin type B to at least 99%.

12. The process of claim 11, wherein said at least one protease administered is selected from the group consisting of: trypsin, immobilized TPCK-trypsin, metalloproteases, endogenous proteases, bacterial proteases, plant derived proteases, and gastric proteases.

13. The process of claim 12, wherein said protease administered is animal free trypsin.

14. The process of claim 11, wherein said at least one dissociating reagent administered is a succinate buffer.

15. The process of claim 11, wherein greater than 90 percent of said botulinum toxin B is nicked.

16. The process of claim 11, wherein approximately about 95 percent to about 100 percent of said botulinum toxin type B is nicked.

17. The process of claim 11, wherein greater than 95 percent of said botulinum toxin B is nicked.

18. The process of claim 11, wherein greater than 99 percent of said botulinum toxin B is nicked.

19. The process of claim 11, wherein greater than 99 percent of said botulinum toxin B is stripped.

20. A method of treating a variety of disorders, comprising administering to a patient in need thereof, a pharmaceutical composition comprising:
(a) activated botulinum toxin type B; and
(b) at least one excipient;
wherein at least 90% of said botulinum toxin type B is nicked; and
wherein at least 99% of said nicked botulinum toxin type B is stripped.

21. The method of claim 20, wherein greater than 90 percent of said botulinum toxin B in said pharmaceutical composition is nicked.

22. The method of claim 20, wherein approximately about 95 percent to about 100 percent of said botulinum toxin type B in said pharmaceutical composition is nicked.

23. The method of claim 20, wherein greater than 95 percent of said botulinum toxin B in said pharmaceutical composition is nicked.

24. The method of claim 20, wherein greater than 99 percent of said botulinum toxin B in said pharmaceutical composition is nicked.

25. The method of claim 20, wherein greater than 99 percent of said botulinum toxin B in said pharmaceutical composition is stripped.

26. The method of claim 20, wherein said at least one excipient of said pharmaceutical composition is selected from the group consisting of: buffers, carriers, stabilizers, preservatives, diluents, vehicles, bulking agents, albumins, gelatins, collagens, proteins, polysaccharides, metals, non-oxidizing amino acid derivatives, and sodium chloride.

27. The method of claim 26, wherein said at least one excipient of said pharmaceutical composition is albumin.

28. The method of claim 26, wherein said at least one excipient of said pharmaceutical composition is a buffer.

29. The method of claim 28, wherein said buffer is a succinate buffer.

30. The method of claim 20, wherein said disorder is selected from the group consisting of:
opthalmologic disorders, neuromuscular diseases, otorhinolaryngological disorders, urogenital disorders, dermatological disorders, pain disorders, inflammatory disorders, secretory disorders, and cutaneous disorders or cosmetic treatment.
